(19) United States
(12) Patent Application Publication
(10) Pub. No.: US 2007/0167633 A1
(43) Pub. Date: Jul. 19, 2007

(54) Process for the preparation of liquid, storage-stable organic isocyanates containing carbodiimide and/or uretonimine groups

(75) Inventors: Stefan Wershofen, Monchengladbach (DE); Marcus Steinwegs, Krefeld (DE); Manfred Schmidt, Dormagen (DE)

Correspondence Address: BAYER MATERIAL SCIENCE LLC, 100 BAYER ROAD, PITTSBURGH, PA, 15205, US

(73) Assignee: Bayer MaterialScience AG

(21) Appl. No.: 11/651,819

(22) Filed: Jan. 10, 2007

(30) Foreign Application Priority Data
Jan. 17, 2006 (DE) 102006002158.4

(51) Int. Cl. C07D 277/20; C07C 267/00

(52) U.S. Cl. 548/202; 560/334

(57) ABSTRACT

The invention relates to a process for the preparation of liquid, storage-stable isocyanate mixtures of low color number and which contain carbodiimide (CD) and/or uretonimine (UI) groups, the isocyanate mixtures obtainable by this process, and to a process for the preparation of blends of these isocyanates with additional isocyanate components and to a process for the preparation of prepolymers containing isocyanate groups and of polyurethane plastics, preferably polyurethane foams.

### CROSS REFERENCE TO RELATED PATENT APPLICATION

The present patent application claims the right of priority under 35 U.S.C. §119 (a)-(d) of German Patent Application No. 10 2006 002 158, filed Jan. 17, 2006.

### BACKGROUND OF THE INVENTION

The invention relates to a process for the preparation of liquid, storage-stable isocyanate mixtures of low color number containing carbodiimide (CD) and/or uretonimine (UI) groups, the isocyanate mixtures obtainable by this process, the preparation of blends from these isocyanate mixtures with additional isocyanates, and to a process for the preparation of prepolymers containing isocyanate groups and of polyurethane plastics, and preferably polyurethane foams.

Isocyanate mixtures containing CD and/or UI groups can be prepared in a simple manner using the highly active catalysts from the phospholine series, and particularly the phospholine oxide series of catalysts. Such isocyanate mixtures are prepared by the processes as described in U.S. Pat. No. 2,853,473, U.S. Pat. No. 6,120,699 and EP-A-515 933.

The high catalytic activity of the phospholine catalysts, and specifically of the phospholine oxide catalysts, on the one hand is desirable in order to start up the carbodiimidization reaction under gentle temperature conditions. However, on the other hand, no process is known to date which ensures effective termination of the phospholine catalysis or phospholine oxide catalysis without limitation. The carbodiimidized isocyanates tend to after-react, i.e. they release gas as a result of evolution of CO₂. This then leads to a build up of pressure, for example, in the storage tanks, and especially at higher temperatures.

There has been no lack of attempts to discover an effective means of terminating the phospholine catalysis. Various terminators are mentioned, for example, in the patent specifications DE-A-25 37 685, EP-A-515 933, EP-A-609 698 and U.S. Pat. No. 6,120,699. These terminators include, for example, acids, acid chlorides, chloroformates, silylated acids and halides of the main group elements. The termination of the phospholine catalysts with acids, which, for example, can also be in the form of acid chlorides, is not sufficiently effective.

According to the teaching of EP-A-515 933, CD/UI-containing isocyanate mixtures prepared by means of phospholine catalysis are terminated with at least an equimolar amount, and preferably from 1 to 2 times the molar amount, based on the catalyst employed, of e.g. trimethylsilyl trifluoromethanesulfonate (TMST). In practice, however, it has been found that CD/UI-containing isocyanates prepared in such a way are of only limited suitability for the preparation of prepolymers, i.e. reaction products of these CD/UI-containing isocyanates with polyols. The correspondingly prepared reaction products of polyols and the CD/UI-modified isocyanates tend to release gas, which can lead to a build up of pressure in the transportation tanks or to foaming during the handling of such products.

This problem can be by-passed by employing the silylated acid to terminate the phospholine catalyst analogously to EP-A-515 933 in higher molar equivalents (e.g. 5:1-10:1, based on the catalyst). In practice, however, it is then found that the resultant CD/UI-modified isocyanates have a significantly poorer color number. This then also applies to the prepolymers prepared therefrom.

This also applies if the phospholine catalyst is terminated with acids of the trifluoromethanesulfonic acid type, in accordance with U.S. Pat. No. 6,120,699. Prepolymers prepared from these CD/UI-modified isocyanates also have a considerably increased color number.

The object of the present invention was therefore to provide a simple and economical process for the preparation of liquid, storage-stable and light-colored isocyanate mixtures containing carbodiimide and/or uretonimine groups which does not have the deficiencies described above and which results liquid, storage-stable isocyanate mixtures of low color numbers.

### SUMMARY OF THE INVENTION

The invention relates to a process for the preparation of organic isocyanates containing carbodiimide and/or uretonimine groups. This process comprises partially carbodiimidizing one or more organic isocyanates having a Hazen color number of ≦100 APHA, preferably ≦50 APHA, with one or more catalysts of the phospholine type; and subsequently terminating the carbodiimidization reaction. In accordance with the present invention, the terminator is added in at least two portions. The temperature of the reaction mixture is higher during the addition of the first portion of terminator than the temperature of the reaction mixture during the addition of the second portion of terminator. This process yields a light-colored isocyanate product.

The present invention also relates to the organic isocyanates containing carbodiimide and/or uretonimine groups which are obtainable by the abovementioned process. These organic isocyanates containing carbodiimide and/or uretonimine groups are liquid at room temperature, and, depending on the CD/UI content and/or on the isocyanate employed, may be liquid down to low temperatures (e.g. 0° C.).

The present invention also provides a process for the preparation of isocyanate blends. These blends comprise the organic isocyanates containing carbodiimide and/or uretonimine groups according to the invention, and at least one other isocyanate component which is different than the isocyanates of the invention which contain carbodiimide and/or uretonimine groups. This invention also provides a process for the preparation of prepolymers which contain isocyanate groups and exhibit an improved color number from the isocyanates containing CD and/or UI groups of this invention.

Finally, the invention also provides a process for the preparation of polyurethane plastics, and preferably polyurethane foams, comprising reacting the organic isocyanates containing carbodiimide and/or uretonimine groups of the invention with at least one isocyanate-reactive component.

### DETAILED DESCRIPTION OF THE INVENTION

As described and used herein, the Hazen color number can be measured in accordance with DIN/EN/ISO 6271-2 (draft of September 2002) in substance against water as the reference, at a layer thickness of 5 cm. For the measuring instrument, a Dr. Lange LICO 300 photometer e.g. can be employed.

Organic isocyanates having a higher color number can, of course, also be used as starting substances. When these higher color number isocyanates are used, however, the advantages with respect of the favorable color values are not utilized to the full extent.

Suitable organic isocyanates to be used as starting materials for the present invention include any desired organic isocyanates which have a Hazen color number of ≦100 APHA, preferably ≦50 APHA. It is preferred that the process according to the invention provides for the carbodiimidization of organic diisocyanates which can in turn be employed in polyurethane chemistry.

Organic isocyanates having a higher color number can, of course, also be used as starting substances. In this case, however, the advantages with respect to the favorable color values cannot be utilized to the full extent.

Suitable isocyanates to be used in accordance with the present invention include, for example, aromatic, araliphatic, aliphatic and/or cycloaliphatic diisocyanates and/or polyisocyanates.

Representatives of the aliphatic and/or cycloaliphatic diisocyanates which may be mentioned by way of example are isophorone-diisocyanate, hexamethylene-diisocyanate and dicyclohexylmethane-diisocyanate. In each case, the pure isomers and/or any desired isomer mixtures may be used herein.

Representatives of the araliphatic diisocyanates which may be mentioned by way of example are the various isomers of xylidene-diisocyanates. Aromatic di- and polyisocyanates, such as toluene-diisocyanate, and di- and polyisocyanates of the diphenylmethane series, are suitable for the starting isocyanate component of the present invention.

In particular, the following isocyanates are suitable starting materials:
aromatic diisocyanates, such as 2,4- and/or 2,6-diisocyanatotoluene (TDI), 2,2′-, 2,4′- and/or 4,4′-diisocyanatodiphenylmethane (MDI) and any desired mixtures of such aromatic diisocyanates;
   and
di- and polyisocyanate mixtures of the diphenylmethane series having a content of monomeric diisocyanatodiphenylmethane isomers of from 80 to 100 wt. % and a content of polyisocyanates of the diphenylmethane series which are more than difunctional of from 0 to 20 wt. %, with the diisocyanatodiphenylmethane isomers being composed of 0 to 100% by weight of 4,4′-diisocyanatodiphenylmethane, 100 to 0% by weight of 2,4′-diisocyanatodiphenylmethane, and 0 to 8% by weight of 2,2′-diisocyanatodiphenylmethane, with the sum of the percentages of the three isomers totaling 100% by weight of the monomer.
   Organic isocyanates which are preferred as starting materials are, in particular, aromatic diisocyanates, such as 2,4- and/or 2,6-diisocyanatotoluene (TDI), 2,2′-, 2,4′- and/or 4,4′-diisocyanatodiphenylmethane (MDI) and any desired mixtures of such aromatic diisocyanates. More preferred starting materials are 2,2′-, 2,4′- and/or 4,4′-diisocyanatodiphenylmethane (MDI) and any desired mixtures of such aromatic diisocyanates, with the sum of 2,2′-, 2,4′- and/or 4,4′-diisocyanatodiphenylmethane in the starting material (organic isocyanate) being at least 85% by weight of the total weight, and the diisocyanatodiphenylmethane isomers being composed of from 0 to 100% by weight of 4,4′-diisocyanatodiphenylmethane, from 100 to 0% by weight of 2,4′-diisocyanatodiphenylmethane and of from 0 to 8% by weight of 2,2′-diisocyanatodiphenylmethane, with the sum of the percentages stated totaling 100% by weight. Most preferred starting materials are 2,2′-, 2,4′- and/or 4,4′-diisocyanatodiphenylmethane (MDI), and any desired mixtures of aromatic diisocyanates, with the sum of 2,2′-, 2,4′- and/or 4,4′-diisocyanatodiphenylmethane in the starting material (i.e. the starting organic isocyanate) being at least 90% by weight, and the diisocyanatodiphenylmethane isomers being composed of 0 to 100% by weight of 4,4′-diisocyanatodiphenylmethane, 100 to 0% by weight of 2,4′-diisocyanatodiphenylmethane and 0 to 8% by weight of 2,2′-diisocyanatodiphenyl-methane, with the sum of the percentages of the three isomers totaling 100% by weight. Most particularly preferred starting materials are 2,2′-, 2,4′- and/or 4,4′-diisocyanatodiphenylmethane (MDI) and any desired mixtures of aromatic diisocyanates, with the sum of 2,2′-, 2,4′- and/or 4,4′-diisocyanatodiphenylmethane present in the starting material (i.e. the starting organic isocyanate) being at least 99% by weight and the diisocyanatodiphenylmethane isomers being composed of 0 to 100% by weight of 4,4′-diisocyanatodiphenylmethane, 100 to 0% by weight of 2,4′-diisocyanatodiphenylmethane, and 0 to 8% by weight of 2,2′-diisocyanatodiphenylmethane, with the sum of the percentages stated for the three isomers totaling 100% by weight.

The process according to the invention is carried out in the presence of catalysts of the phospholine type. The catalysts of the phospholine type are known and described in, for example, EP-A-515 933 and U.S. Pat. No. 6,120,699, the disclosures of which are hereby incorporated by reference. Typical examples of these catalysts are, for example, the mixtures, known from the prior art, of the phospholine oxides which correspond to the formulas:

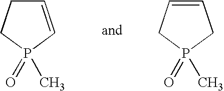

The amount of catalyst employed depends on the quality and/or the reactivity of the starting isocyanates. Thus, the specific amount of catalyst needed can most easily and readily be determined in a preliminary experiment.

The carbodiimidization reaction is conventionally carried out in the temperature range between 50 to 150° C., preferably from 60 to 100° C. However, significantly higher reaction temperatures are also possible (i.e. up to approx. 280° C.). The optimum reaction temperature for the carbodiimidization reaction depends on the nature of the starting isocyanates and/or of the catalyst employed, and can be determined in a simple preliminary experiment.

The carbodiimidization reaction is, in general, interrupted when a degree of carbodiimidization of from 3 to 50%, and preferably from 5 to 30%, is reached. The phrase “the degree of carbodiimidization” refers to the percentage of carbodiimidized isocyanate groups, with respect to the total amount of isocyanate groups present in the starting isocyanate.

The degree of carbodiimidization can be determined while the process according to the invention is being carried out, by determination of the % NCO by, for example, means of titration, which is known per se to the person skilled in the art, or by means of suitable online methods. A suitable online method is, for example, near infra-red or middle infra-red analysis.

The degree of carbodiimidization can likewise be ascertained while the process according to the invention is being carried out, for example, from the amount (i.e. quantity) of carbon dioxide escaping in the reactor mixture. This amount of carbon dioxide, which can be determined volumetrically, thus provides information about the degree of carbodiimidization reached at any point in time.

Furthermore, in principle, other suitable offline or online methods of process monitoring which are known to the person skilled in the art can also be employed.

To end the carbodiimidization reaction, it is preferable to add at least the equimolar amount, more preferably a 1- to 20-fold molar excess, and most preferably a 1- to 10-fold molar excess, based on the weight of the catalyst, of a terminator or an alkylating agent. A mixture of terminators may also be employed. A preferred catalyst terminator is trimethylsilyl trifluoromethanesulfonate (TMST). In this context, an alkylating agent or trimethylsilyl trifluoromethanesulfonate (TMST) is preferably employed as the sole terminator.

Preferred alkylating agents are esters of trifluoromethanesulfonic acid, esters of inorganic acids (preferably strong inorganic acids) or trialkyloxonium compounds.

In accordance with the present invention, the carbodiimidization reaction is terminated by adding the terminator in at least two portions, with the temperature of the reaction mixture during the addition of the first portion being higher than the temperature of the reaction mixture during the addition of the second portion. Preferably, when the addition of the terminator occurs in more than two portions, the temperature of the reaction mixture during the addition of the first portion is higher than the temperature of the reaction mixture during the addition of all further or later added portions. It is preferred that the temperature of the reaction mixture during the addition of the first portion of the terminator is at least 20° C., and more preferably at least 30° C. higher than the temperature of the reaction mixture during the addition of the second portion of terminator, and preferably also of all further or later added portions of terminator.

Preferably, the termination of the carbodiimization reaction is carried out by addition of the terminator in two to five portions, and more preferably in two, three or four portions. The first portion of terminator is preferably present in an amount such that at least the molar amount of terminator which corresponds to the molar amount of catalyst that was added. In the second and the optionally further portions of terminator addition, additional terminator is then added. The second and any later added portions of terminator obviously result in a total quantity of terminator which exceeds the molar amount of catalyst that was added.

Preferably, there is a time interval of at least 1 minute, more preferably of at least 5 minutes, and most preferably of at least 10 minutes between the addition of the first portion of terminator and the addition of the second portion of terminator. The reaction product of the carbodiimidization reaction can contain color stabilizers such as those which are conventionally added to isocyanates. In this context, the point in time of the addition of the stabilizers is not critical. The color stabilizers can be added either to the isocyanate which is used as the starting material, before the carbodiimidization, or to the reaction product when the carbodiimization reaction has ended. Likewise, it is possible to add color stabilizers to both the starting material and to the reaction product. Such stabilizers are generally known to the person skilled in the art and include e.g. substances from the group consisting of sterically hindered phenols, phosphorous acid esters or sterically hindered amines. The color stabilizers can in each case be employed by themselves or in a mixture with other representatives of the same or different substance groups. The amounts of color stabilizers employed varies in the order of magnitude known to the person skilled in the art, conventionally in the range of from 100 ppm to 10,000 ppm for the individual substance or the mixture, based on the total weight of the isocyanate used as the starting material or of the reaction product of the carbodiimidization.

Prepolymers containing isocyanate groups are obtained by, for example, reaction of the organic isocyanates containing carbodiimide and/or uretonimine groups which are prepared by the process of the present invention with one or more conventional polyols which are known to be suitable in polyurethane chemistry. Suitable polyols include both simple polybasic alcohols having a molecular weight in the range of from 62 to 599 g/mol, preferably 62 to 300 g/mol, such as e.g. ethylene glycol, trimethylolpropane, propane-1,2-diol, butane-1,2-diol or butane-2,3-diol, hexanediol, octanediol, dodecanediol and/or octadecanediol, and in particular, higher molecular weight polyether polyols and/or polyester polyols of the type known per se from polyurethane chemistry which have molecular weights of from 600 to 8,000 g/mol, preferably 800 to 4,000 g/mol. Such higher molecular weight compounds typically contain at least two, and as a rule from 2 to 8, and preferably from 2 to 4 primary and/or secondary hydroxyl groups. Examples of such polyols are described in, for example, U.S. Pat. No. 4,218,543, at column 7, line 29 to column 9, line 32, the disclosure of which is hereby incorporated by reference.

The advantages of the process according to the invention are readily apparent. Due to the addition of the terminator in two or more portions to the reaction mixture at a different temperatures, both the isocyanates containing carbodiimide and/or uretonimine groups and the prepolymers prepared therefrom have a light color.

These organic isocyanates containing carbodiimide and/or uretonimine groups and the prepolymers prepared therefrom by reaction of the isocyanates of the invention with polyols are valuable starting materials for the preparation of polyurethane plastics by the reaction of the isocyanates of the invention or prepolymers thereof with one or more polyols (e.g. polyether polyols and/or polyester polyols) by the isocyanate polyaddition process.

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

The following starting substances were used in the working examples:
Isocyanate A: 4,4′-diphenylmethane diisocyanate having an NCO group content of 33.6% by weight (Desmodur 44M®, Bayer AG)
Catalyst A: a technical-grade mixture of 1-methyl-1-oxo-1-phosphacyclopent-2-ene and 1-methyl-1-oxo-1-phosphacyclopent-3-ene, 1 wt. % strength in toluene
Terminator A: trifluoromethanesulfonic acid ethyl ester (TFMSEE)
Terminator B: trimethylsilyl trifluoromethanesulfonate (TMST)

The following general instructions were used for the preparation of the organic isocyanate containing carbodiimide and/or uretonimine groups:

10 kg of Isocyanate A having a Hazen color number of <15 APHA, which contained 750 ppm 3,5-di-tert-butyl-4-hydroxytoluene, were heated to approx. 90° C. under N₂/while stirring. 2.5 ppm of catalyst was then added in the form of a 1% strength solution in toluene. The reaction mixture was heated at approx. 95° C. under N₂/while stirring until the desired NCO content was reached. Thereafter, the carbodiimidization reaction was terminated by the addition of 10 ppm the specific terminator (i.e. trifluoromethanesulfonic acid ethyl ester (TFMSEE) or trimethylsilyl trifluoromethanesulfonate (TMST); see table for specific details) the mixture was subsequently stirred for 1 hour. A second amount of terminator (see table) was added at room temperature.

The results are summarized in the following table.

The Hazen color number was measured in accordance with DIN/EN/ISO 6271-2 (draft of September 2002), in substance against water as the reference at a layer thickness of 5 cm. A Dr. Lange LICO 300 photometer was employed as the measuring instrument.

| | Educt | | | | Product | |
|---|---|---|---|---|---|---|
| | Total terminator concentration | | First amount of | Second amount of | NCO | Hazen |
| | [ppm] | Terminator | terminator [ppm] | terminator [ppm] | value [%] | [APHA] |
| Comparison Example 1 | 50 | TMST | 50 | — | 29.3 | 529 |
| Comparison Example 2 | 50 | TFMSEE | 50 | — | 29.6 | 217 |
| Example 1 | 50 | TMST | 10 | 40 | 29.0 | 78 |
| Example 2 | 50 | TFMSEE | 10 | 40 | 29.8 | 69 |

The examples according to the invention illustrate the advantageous effect of the multi-stage addition of terminator with respect of the product color in comparison to the comparison examples in which all of the terminator was added in one step.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the preparation of organic isocyanates containing carbodiimide and/or uretonimine groups, comprising
(A) partially carbodiimidizing
(1) one or more organic isocyanates having a Hazen color number of <100 APHA,
in the presence of
(2) one or more catalysts of the phospholine type
(B) adding a first portion of a terminator to the reaction mixture from (A) and
(C) adding a second portion of a terminator to the reaction mixture from (B), wherein the temperature of the reaction mixture during the addition of the first portion of terminator is higher than the temperature of the reaction mixture during the addition of the second portion of terminator.

2. The process of claim 1, wherein (1) said one or more organic isocyanates have a Hazen color number of ≦50 APHA,

3. The process of claim 1, additionally comprising (D) adding a third portion of a terminator to the reaction mixture from (C), wherein the temperature of the reaction mixture during the addition of the third portion of terminator is lower than the temperature of the reaction mixture during the addition of the first portion of terminator.

4. The process of claim 1, wherein the temperature of the reaction mixture during the addition of the first portion of the terminator is at least 20° C. higher than the temperature of the reaction mixture during the addition of the second portion of terminator.

5. The process of claim 1, wherein the temperature of the reaction mixture during the addition of the first portion of the terminator is at least 30° C. higher than the temperature of the reaction mixture during the addition of the second portion of terminator.
